(19) United States
(12) Patent Application Publication
(10) Pub. No.: US 2017/0164856 A1
(43) Pub. Date: Jun. 15, 2017

(54) SENSING OF A USER'S PHYSIOLOGICAL CONTEXT USING A HAND-HELD DEVICE

(71) Applicant: Intel Corporation, Santa Clara, CA (US)

(72) Inventors: Amit S. Baxi, Thane (West) (IN); Vincent S. Mageshkumar, Navi Mumbai (IN)

(21) Appl. No.: 14/966,254

(22) Filed: Dec. 11, 2015

(51) Int. Cl. A61B 5/0404; A61B 5/0492; A61B 5/0205

(52) U.S. Cl. CPC A61B 5/0404; A61B 5/0205; A61B 5/0492

(57) ABSTRACT

Embodiments of the present disclosure provide techniques and configurations for an apparatus for opportunistic measurements of a user's physiological context. In one instance, an apparatus may comprise a hand-held device, and include a surface that includes two or more electrodes disposed on the surface to contact with portions of the user's hands when the hands are in contact with the surface while the user holds the hand-held device, to obtain signals to be used to determine a physiological context of the user. The device may further include circuitry coupled with the two or more electrodes to identify at least two of the electrodes that are in contact with the user's respective portions of hands and, on identification, collect the signals to determine the physiological context using the identified electrodes while the contact between the portions of the hands and the identified electrodes is maintained. Other embodiments may be described and/or claimed.

### FIELD

Embodiments of the present disclosure generally relate to the field of sensor devices, and more particularly, to sensor devices for providing opportunistic measurements of a user's physiological context.

### BACKGROUND

Sensing of a user's physiological context may provide continuous information about the user's biological and mental state, behavior and preferences so long as the user is using the device for other everyday needs. This information may be used by context-aware applications for personalization related to a user's state of health, such as coaching, alerting, chronic disease management, personalized medicine, and the like. Today's proliferation of hand-held computing/communication devices may provide opportunity for sensing of a user's physiological context. However, determination of the user's physiological context on the user's demand may consume a substantial amount of the user's time and effort and may require using substantial energy, hardware, and computing resources.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example and not by way of limitation in the figures of the accompanying drawings.

FIG. 1 is an example block diagram illustrating an apparatus for opportunistic measurements of a user's physiological context, incorporated with the teachings of the present disclosure, in accordance with some embodiments.

FIGS. 2 and 3 illustrate example front and rear views of a hand-held device 200 for opportunistic measurements of a user's physiological context, in accordance with some embodiments.

FIG. 4 is an example schematic diagram illustrating circuitry of the apparatus of FIG. 1, in accordance with some embodiments.

FIG. 5 is an example process flow diagram for opportunistic measurements of a user's physiological context, in accordance with some embodiments.

FIG. 6 is an example block diagram for adaptive noise cancellation or reduction in a signal indicative of the user's physiological context, in accordance with some embodiments.

FIG. 7 illustrates example views of a hand-held device configured with measurements of a user's physiological context, in accordance with some embodiments.

### DETAILED DESCRIPTION

Embodiments of the present disclosure include techniques and configurations for opportunistic measurements of a user's physiological context using a hand-held computing/communication device (hereinafter, simply hand-held device). Opportunistic measurements may include measurements of the user's physiological context, e.g., during the user's interaction with the device, when at least portions of one or more user's hands (e.g., fingers, palms, and/or wrists) are disposed on a surface of the device to hold, or otherwise interact with, the device.

In accordance with embodiments, an apparatus for opportunistic measurements of a user's physiological context, such as a hand-held device, may include a surface that includes two or more electrodes disposed on the surface to contact with at least portions of the user's one or more hands when the one or more hands are in contact with the surface while the user holds the hand-held device, to obtain signals to be used to determine a physiological context of the user. The device may further include circuitry coupled with the two or more electrodes to identify at least two of the electrodes that are in contact with the user's respective portions of one or more hands and, on identification, collect the signals to determine the physiological context using the identified electrodes while the contact between the portions of the one or more hands and the identified electrodes is maintained.

In the following detailed description, reference is made to the accompanying drawings that form a part hereof, wherein like numerals designate like parts throughout, and in which are shown by way of illustration embodiments in which the subject matter of the present disclosure may be practiced. It is to be understood that other embodiments may be utilized and structural or logical changes may be made without departing from the scope of the present disclosure. Therefore, the following detailed description is not to be taken in a limiting sense, and the scope of embodiments is defined by the appended claims and their equivalents.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B, and C).

The description may use perspective-based descriptions such as top/bottom, in/out, over/under, and the like. Such descriptions are merely used to facilitate the discussion and are not intended to restrict the application of embodiments described herein to any particular orientation.

The description may use the phrases “in an embodiment,” or “in embodiments,” which may each refer to one or more of the same or different embodiments. Furthermore, the terms “comprising,” “including,” “having,” and the like, as used with respect to embodiments of the present disclosure, are synonymous.

The term “coupled with,” along with its derivatives, may be used herein. “Coupled” may mean one or more of the following. “Coupled” may mean that two or more elements are in direct physical, electrical, or optical contact. However, “coupled” may also mean that two or more elements indirectly contact each other, but yet still cooperate or interact with each other, and may mean that one or more other elements are coupled or connected between the elements that are said to be coupled with each other. The term “directly coupled” may mean that two or more elements are in direct or indirect contact.

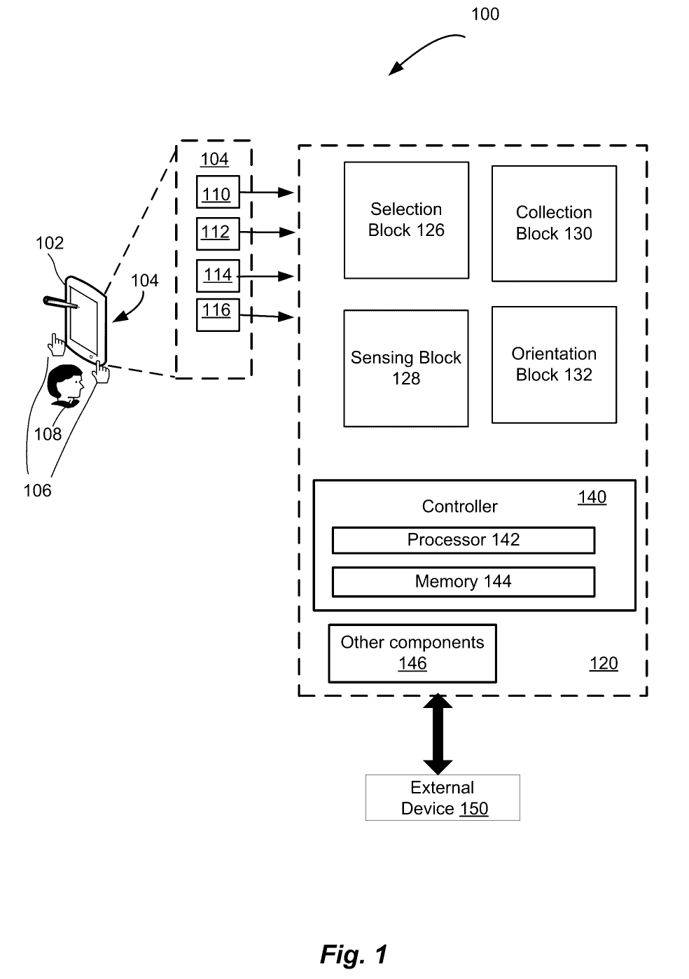

FIG. 1 is an example block diagram illustrating an apparatus 100 for opportunistic measurements of a user's physiological context, incorporated with the teachings of the present disclosure, in accordance with some embodiments. In embodiments, the apparatus 100 may comprise a hand-held device 102, such as a tablet computer, a smartphone, or the like. The apparatus 100 may comprise a surface 104, e.g., a portion of a back side, a bezel, or other part of the hand-held device 102. The surface 104 may include two or more sensors (e.g., electrodes) 110, 112, 114, 116 disposed on the surface 104. The electrodes 110, 112, 114, 116 may be configured to enable contact with portions of one or more hands 106 of a user 108, e.g., fingers, palms, or wrists, when the user's portions of one or more hands 106 are at least partially disposed on the surface 104 to hold or interact with the hand-held device 102, to obtain signals indicative of a user's physiological context.

The electrodes 110, 112, 114, 116 may provide readings related to various user body functions as discussed below in greater detail. For example, electrodes 110, 112, 114, 116 may be electrically conductive electrodes. Electrodes 110, 112, 114, 116 may be configured to measure electrocardiogram (ECG) bio-potentials from the user's hands 106. In embodiments, electrodes 110, 112, 114, 116 may be configured to measure electromyographic (EMG) data, photoplethysmographic (PPG) data, or the like. In embodiments, the sensors 110, 112, 114, 116 may further include temperature sensors, sweat chemical sensors, motion sensors, galvanic skin response (GSR) sensors, piezo crystals, pressure sensors, or the like. For example, to measure PPG data, optical sensors may be coupled with (e.g., embedded in or under) the electrodes 110, 112, 114, 116. A number of electrodes illustrated in FIG. 1 are provided for illustration purposes only and are not limiting this disclosure. Different types of sensors providing readings of a user's physiological context may be disposed on (e.g., embedded in) the surface 104 of the hand-held device 102.

The apparatus 100 may further comprise electronic circuitry 120 coupled with the electrodes 110, 112, 114, 116, to identify those of the electrodes 110, 112, 114, 116 that are in contact with the respective portions of the one or more hands 106 of the user 108 while the user 108 holds the hand-held device 102. Once the electrodes in contact with the users' portions of one or more hands 106 are identified, the circuitry 120 may use the identified electrodes to collect the signals to determine a physiological context, while the contact between the portions of the user's one or more hands 106 and the identified electrodes is maintained, thus providing opportunistic measurements of the user's physiological context.

More specifically, the circuitry 120 may include a selection block 126 configured to sequentially select, on a continuous or periodic basis, pairs of electrodes from the electrodes 110, 112, 114, 116 and to cause a test current flow through selected pairs of electrodes, to identify a pair of electrodes that form a closed circuit with the one or more hands 106 and the body of the user 108. The identified pair of electrodes may be in contact with the user's portions of one or more hands 106, to provide opportunistic sensing of the signals indicative of the physiological context of the user.

The circuitry 120 may further include a sensing block 128 coupled with block 126 and configured to detect whether a current flows through the body of the user 108 in response to the injection of the test current, to identify the pair of electrodes that may be in contact with the user's portions of one or more hands 106.

The circuitry 120 may further include a collection block 130 coupled with the selection block 126 and configured to collect the readings indicative of the user's physiological context from the identified electrodes.

The circuitry 120 may further include an orientation block 132 configured to determine orientation of the hand-held device 102 relative to the user's one or more hands 106. Based on the orientation of the hand-held device 102, an order of holding by the user's portions of one or more hands 106 of the hand-held device 102 relative to the identified electrodes may be determined. For example, it may be determined that a left hand is in contact with one of the identified electrodes (e.g., 110), and a right hand is in contact with another of the identified electrodes (e.g., 112). The respective electrodes may be connected to corresponding (e.g., positive or negative) inputs of the collection block 130, in order provide correct measurements of the user's physiological context, such as ECG signals.

The apparatus 100 may further include a processing unit (e.g., controller) 140 coupled with selection block 126, sensing block 128, collection block 130, and orientation block 132, and configured to operate the electrode selection, identification, and connection to the circuitry 120 as briefly describe above, and process the readings provided by the identified ones of electrodes 110, 112, 114, and 116.

In some embodiments, the controller 140 may include a processor 142 configured to operate blocks 126, 128, 130, and 132, and process the readings (signals) provided to the circuitry 120. The controller 140 may further include memory 144 having instructions that, when executed on the processor 142, may cause the processor 142 to perform signal processing as described above. The processor 142 may be implemented as having multi-cores, e.g., a multi-core microprocessor. Memory 144 may be temporal and/or persistent storage of any type, including, but not limited to, volatile and non-volatile memory, optical, magnetic, and/or solid state mass storage, and so forth.

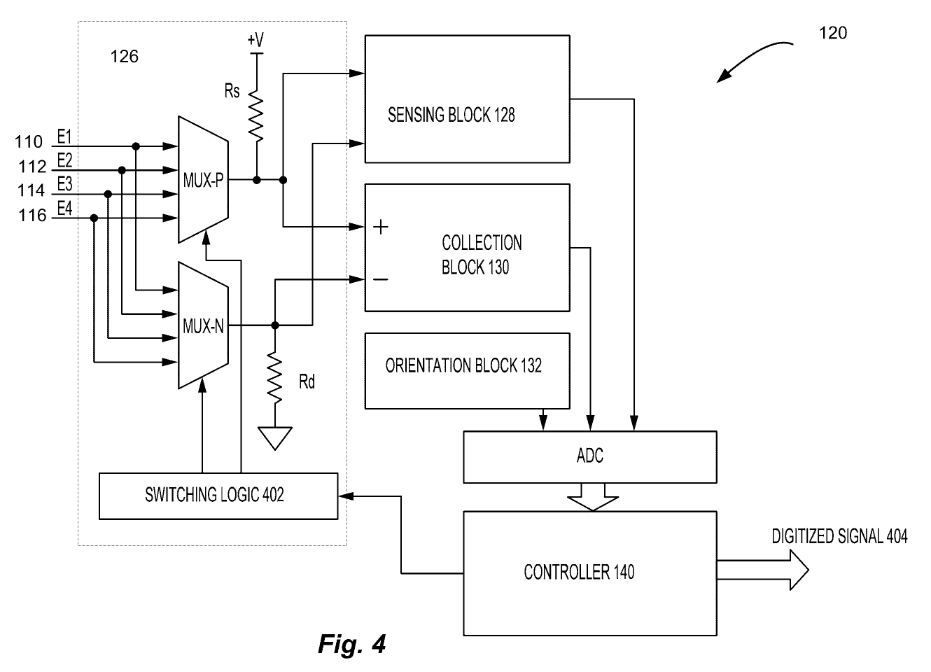

The circuitry 120 may include other components 146 necessary for the functioning of the apparatus 100. For example, other components 146 may include a transceiver to communicate the user's physiological context measurements over one or more wired or wireless network(s) with any other suitable device, such as external computing device 150. In embodiments, the controller 140 may comprise a system on chip (SoC) or system in package (SiP). In embodiments, the circuitry 120 may be disposed on a printed circuit board (PCB) communicatively coupled with the sensors 110, 112, 114, 116. The operation of circuitry 120 will be described in greater detail in reference to FIGS. 4-6.

In order to allow for seamless opportunistic sensing of the user's physiological context, the user may need to have access to the electrodes providing measurements of the user's physiological context in natural positions and during regular user activities, such as during operation of a hand-held device. Accordingly, the electrodes may be placed in various portions of a hand-held device with which the user may come in direct or indirect contact, depending on a type of a device. For example, the sensors may be placed in various parts of a casing of a hand-held device.

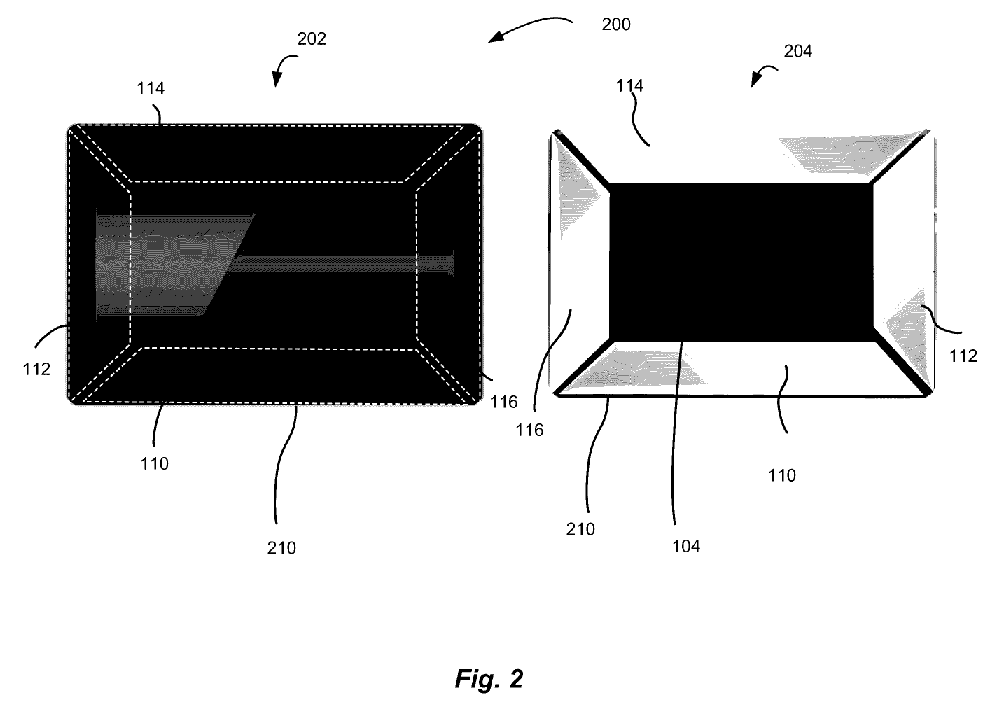

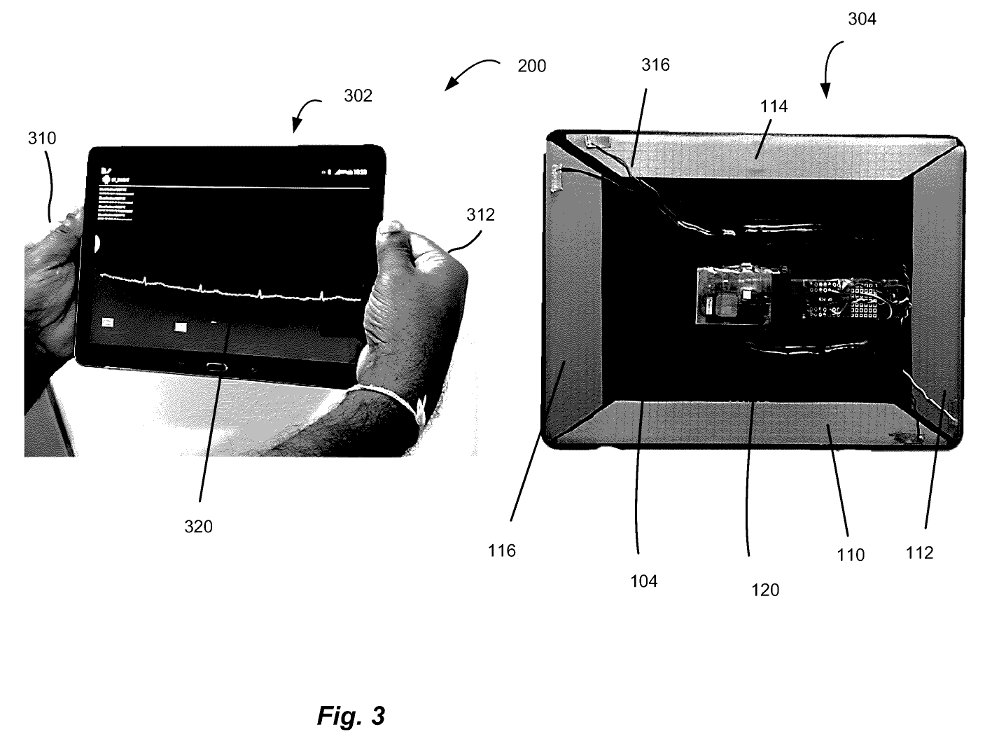

FIGS. 2 and 3 illustrate example front and rear views of a hand-held device 200 for opportunistic measurements of a user's physiological context, in accordance with some embodiments. The hand-held device 200 may include one or more components of the apparatus 100 of FIG. 1 described above and enumerated with like numerals for simplicity purposes.

As described in reference to FIG. 1, the hand-held device 200 (e.g., a tablet computer) may have a casing 210. The casing 210 may include a surface 104, in this example, a back surface of the hand-held device 200, which may come in direct contact with the user's one or more hands (not shown) when the user operates (e.g., holds) the hand-held device 200. The device 200 may include an array of electrodes 110, 112, 114, and 116 disposed on the casing 210, as shown in dotted lines in the front view 202 of the device 200 and in more detail in the rear view 204 of the device 200. As shown, the electrodes 110, 112, 114, 116 may be disposed substantially around the perimeter of the back surface 104 of the hand-held device 200, as shown in rear view 204 of the device 200. The electrodes 110, 112, 114, 116 may be made of, for example, a metallic film (e.g., conductive copper polymer), or may be non-metallic, e.g., may be made of conductive screen, printed conductive ink, conductive fabric or conductive elastomer.

As shown, the electrodes 110, 112, 114, 116 may have trapezoid-like shapes, to extend toward edges of the surface of the hand-held device to provide the contact of the user's portions of the one or more hands with the identified electrodes when the tablet is held in either portrait or landscape modes. It will be understood that the number, size, shape, and placement of electrodes may vary depending on the hand-held device and may be chosen to raise the probability of contact with both hands when the user is casually interacting with the device, for example, when the device is held by the edges, or by the back, or any combination of the above.

Two of the electrodes 110, 112, 114, 116 may be identified as being in contact with the user's portions of one or more hands and used to sense the user's physiological context, e.g., PPG bio-potential from portions of left hand 310, or ECG bio-potentials from left and right portions of hands 310, 312 (e.g., fingers, palms or wrists) of the user, as shown in front view 302 of the device 200 in FIG. 3. For example, as shown in the front view 302 in FIG. 3, on detection of the ECG bio-potential, the device 200 may display an ECG signal 320 that is sensed from a user holding the device 200.

As shown in rear view 304 in FIG. 3, circuitry 120 may be attached to the back surface of the device 200 and may be electrically coupled with the electrodes 110, 112, 114, 116 by means of wires 316.

FIG. 4 is an example schematic diagram illustrating circuitry 120 of the apparatus 100, in accordance with some embodiments. In embodiments, circuitry 120 may be implemented as a hardware amplifier front-end circuit and may be electrically coupled with the electrodes 110, 112, 114, 116 of the apparatus 100 (e.g., hand-held device 200) as shown in FIG. 3. For simplicity purposes, the components of circuitry 120 in FIGS. 1 and 4 are indicated with like numerals.

The circuitry 120 may implement an example technique to measure hand-to-electrode electrical contact impedance (henceforth referred to as electrode-tissue impedance (ETI)) between any combinations of these electrodes in real- or near-real time. Based on the measured impedance (or corresponding voltage value), the electrodes in contact with the user's portions of hands may be identified.

More specifically, a test current (e.g., about 10 microamperes or less) may be injected in each of the electrodes 110, 112, 114, 116 (for example, in 110). Current may be sensed individually from each of the remaining electrodes (for example, 112, 114, 116). Alternatively, a corresponding voltage may be sensed between the electrodes (e.g., between 110 and each of 112, 114, or 116). Unless the user's hands make contact with at least two of the electrodes 110, 112, 114, 116, the circuit may remain open and no current may flow between the electrodes. When the user holds the hand-held device and one or both of her hands contact at least two electrodes, the circuit may close (through the user's body) and the current may flow from one electrode to another. If the current (or voltage) between the electrodes is not detected, the test current may be injected into the next electrode (e.g., 112) and corresponding current (or absence thereof) may be sensed in remaining electrodes (e.g., 110, 114, and 116). This operation may continue in a round-robin manner until the closed circuit is identified. Thus, a pair of electrodes in contact with the user's portions of one or more hands may be identified and used for opportunistic measurements of a signal that may indicate the user's physiological context (e.g., PPG or ECG).

As shown, four electrodes 110, 112, 114, 116 may be connected (E1, E2, E3 and E4 respectively) to the inputs of two analog multiplexers, MUX-P and MUX-N comprising in part selection block 126. MUX-P may be configured to connect any one of the electrodes 110, 112, 114, 116 to its output. Similarly, MUX-N may select and connect any one of the electrodes 110, 112, 114, 116 to its output. The output of MUX-P may connect to an input (e.g., positive input) of the collection block 130 (e.g., an amplifier). The output of MUX-N may connect to another (e.g., negative) input of the collection block 130. MUX-P and MUX-N outputs may also connect to the sensing block 128 for sensing ETI. The switching of MUX-P and MUX-N may be operated by the controller 140 using, e.g., switching logic 402, which may implemented as software executable on the processor 142 of the controller 140, or hardware (e.g., firmware). Accordingly, any combination of electrode pairs may be connected to the input of the collection block 130 by the controller 140.

The controller 140 may be further configured to sample the ETI signal and the signal indicating physiological context (e.g., ECG), and determine orientation of the hand-held device. As shown, the signals outputted by blocks 128, 130, and 132 may be fed to the analog to digital converter (ADC) and inputted in a digital form to the controller 140. The controller 140 may process the inputted signals and output a processed digitized signal 404, to be used to determine the user's physiological context.

This described circuit arrangement may provide for injection of a test current in any of the electrodes 110, 112, 114, 116 via a high impedance source (Rs), and detection of the current flowing through the human body via any of the remaining three electrodes in response to the injection of the test current, using a high impedance sink (Rd). Thus, under control of the controller 140, any one electrode may be dynamically configured to inject current into the user's body and any one electrode may be configured to detect current flowing through the user's body, thereby enabling the identification of a pair of electrodes that are making contact with the user's body, for example, with the user's portions of hands.

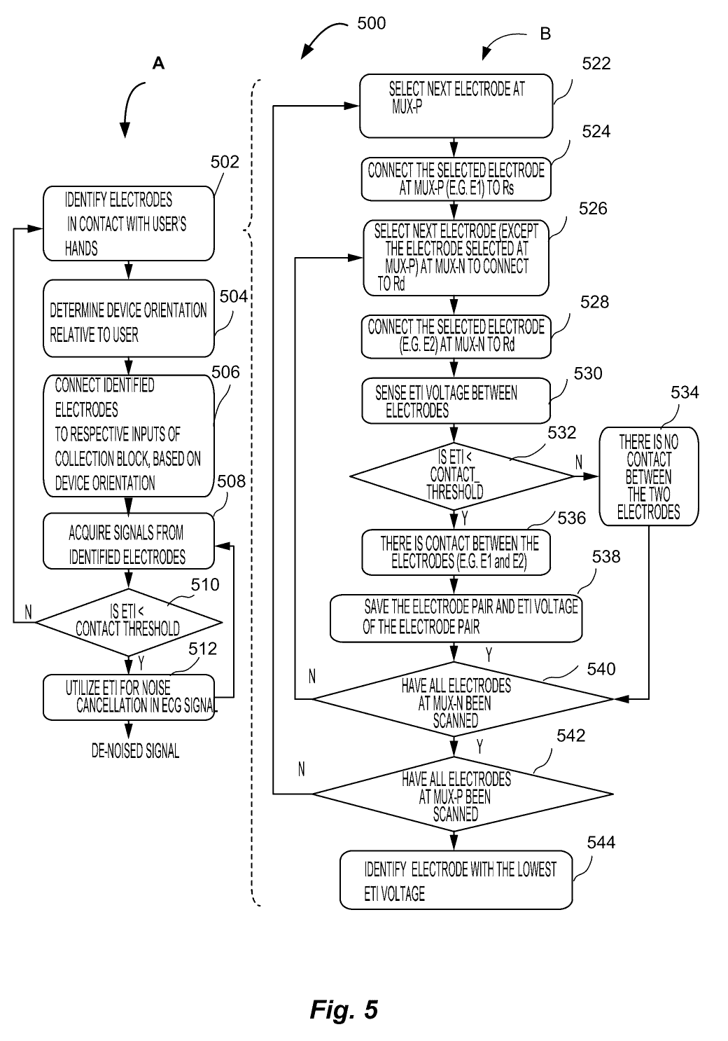

FIG. 5 is an example process flow diagram 500 for opportunistic measurements of a user's physiological context, in accordance with some embodiments. Flowchart A describes an example process for opportunistic sensing of the user's physiological context, such as ECG. Flowchart B describes an example process of block 502 of flowchart A in detail. Specifically, flowchart B explains the method of automatically identifying a pair of electrodes (among existing electrodes, such as 110, 112, 114, 116) with the desired contact with the user's hands, so as to get the desired ECG signal quality. The process 500 described in flow diagrams A and B may be performed by circuitry 120 of the apparatus 100, e.g., hand-held device of FIG. 1. While the process 500 provides an example of measuring signals indicative of ECG, any other suitable physiological context of a user may be measured using the process 500.

At block 502, the process 500 may include identifying a pair of electrodes (e.g., among the electrodes disposed on a surface of a hand-held device as described above) that make contact with the user's portions of one or more hands. The quality of ECG signals provided by the electrodes may depend on the quality of contact of both of the user's hands (fingers, palms, or wrists) with the electrodes. Accordingly, a pair of electrodes may be identified that makes the desired, e.g., “best” contact with the user's portions of hands among the tested electrodes. The “best” contact may be characterized by the lowest contact impedance (e.g., lowest ETI voltage) between the electrodes that may be measured in response to contact of the electrodes with the user's portions of hands.

Flowchart B describes the process of block 502 in detail. As described above, a test current may be injected in any one electrode and the current from other electrodes may be sensed. If there is no contact of the user's hands with the electrodes, no current between the electrodes may flow and ETI voltage may remain above a threshold (e.g., high value voltage, blocks 532 and 534). If there is a contact between the user's hands and the electrode pair, the current may flow through the body of the user and ETI voltage between the electrode pair may be lower than a determined threshold (CONTACT THRESHOLD, blocks 532 and 536).

According to flowchart B, the electrodes may be continuously (or periodically) scanned to determine if ETI between any two electrodes is less than CONTACT THRESHOLD. To accomplish that, one electrode may be selected and connected to current source (Rs) using MUX-P (e.g., successively, in a round robin manner, blocks 522, 524, and 542). The remaining electrodes may be selected and successively connected (in a round robin manner) to Rd using MUX-N (blocks 526, 528, and 540) to sense current (and ETI voltage) flowing through these electrodes (block 530). For any electrode pair, if the ETI voltage is less than CONTACT THRESHOLD, the electrode identifiers and corresponding ETI value may be stored in memory (e.g., 144 of FIG. 1, block 538). Once a scanning cycle is completed (at block 542), an electrode pair with the lowest ETI voltage may be identified at block 544, which may correspond to the “best” or desired contact assuming there is more than one pair with a corresponding ETI value below CONTACT THRESHOLD. The electrode scanning operation may be done continuously or periodically in order to keep track of dynamic handling of the device by the user.

According to standard medical convention, the user's hands may be connected to the circuitry 120 in a particular order, to obtain a correct reading of the ECG signal from the user. More specifically, the left hand of the user may be connected to the positive input of the collection block 130 (e.g., ECG amplifier) and the right hand of the user may be connected to the negative input of the collection block 130 in order to obtain the proper ECG waveform.

Referring again to flowchart A, once the electrode pair with contact to the user's hands has been identified, at block 504, an orientation of the hand-held device relative to the user may be determined by orientation block 132 of the circuitry 120. For example, an orientation sensor (usually integrated inside a hand-held device, such as a tablet computer or a smartphone) may be used to determine which electrode among the identified pair is oriented towards what side of the user's body.

At block 506, identified electrodes may be connected to respective inputs of the collection block 130 (ECG amplifier). For example, the left side electrode may be connected to the positive input of the ECG amplifier, using MUX-P. The right side electrode may be connected to the negative input of the ECG amplifier, using MUX-N.

After identification of the electrodes, determination of their orientation, and provision of corresponding connections with desired polarity to the ECG amplifier, ECG and ETI signals may be sampled (e.g., continuously or periodically) at block 508. To ensure that the skin contact between the selected electrode pair and the user's portions of hands is at the desired or acceptable level, it may be determined at decision block 510 (e.g., at each sample) whether the ETI voltage (between the electrode pair) is still less than CONTACT THRESHOLD. If the ETI voltage is less than CONTACT THRESHOLD, the process 500 may return to block 508 (via block 512 described below) to continue receiving ECG and ETI signals from the identified electrodes. If the ETI voltage is equal to or greater than CONTACT THRESHOLD, such as if the electrode-skin contact is not on a desirable level (e.g., poor or broken), the process may move back to block 502, to identify a new electrode pair for ECG sensing.

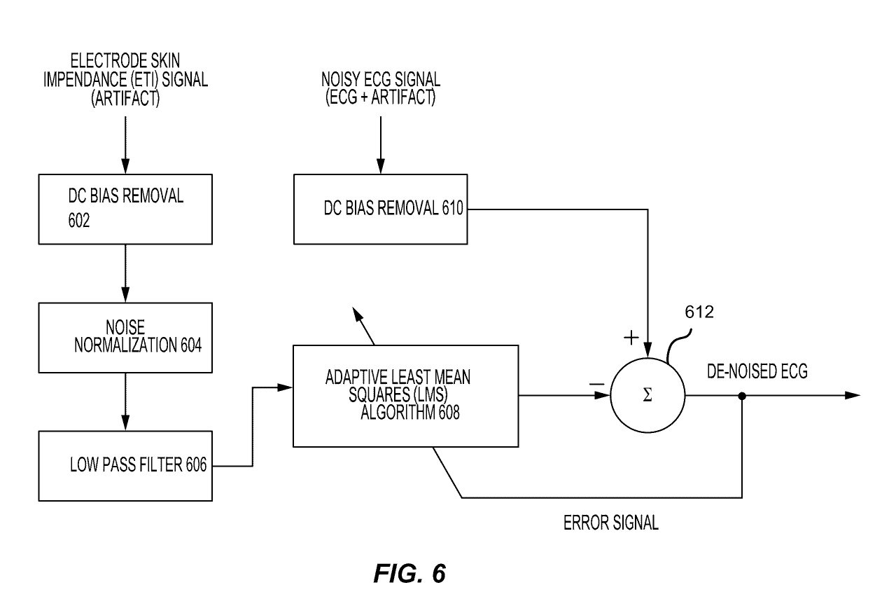

When the user holds the hand-held device, there may be intermittent changes in the contact pressure on the electrodes, which may induce artifacts (noise) in the sensed ECG signal. The ETI signal may track the induced noise and, accordingly, noise from the sensed ECG signal may be reduced or eliminated if ETI is subtracted from the ECG signal. At block 512, the noise cancellation or reduction procedure may be performed on the sensed ECG signal as described in reference to FIG. 6.

FIG. 6 is an example block diagram for adaptive noise cancellation or reduction in a signal indicative of the user's physiological context, in accordance with some embodiments. It will be understood that the described scheme is one example of noise cancelation techniques; other noise cancellation techniques may be applied in the embodiments of this disclosure.

As described above, ETI and ECG signals may be sensed using circuitry 120. In the example illustrated in FIG. 6, a least-mean squares (LMS) based adaptive noise cancellation scheme may be used to recover the ECG signal. The noise cancellation scheme illustrated by the diagram 600 may rely on conditioning the input signals. For example, ETI signal and ECG signal (with noise artifacts) may be conditioned by removal of direct current (DC) bias (blocks 602 and 610 respectively). The ETI signal (e.g., the artifact signal) may be normalized in proportion to the noise in the ECG signal (block 604) and filtered by the low pass filter (block 606). The ETI signal may be subtracted from the ECG signal with noise artifacts at block 612, utilizing an error minimization feedback loop (block 608), to obtain a de-noised ECG signal.

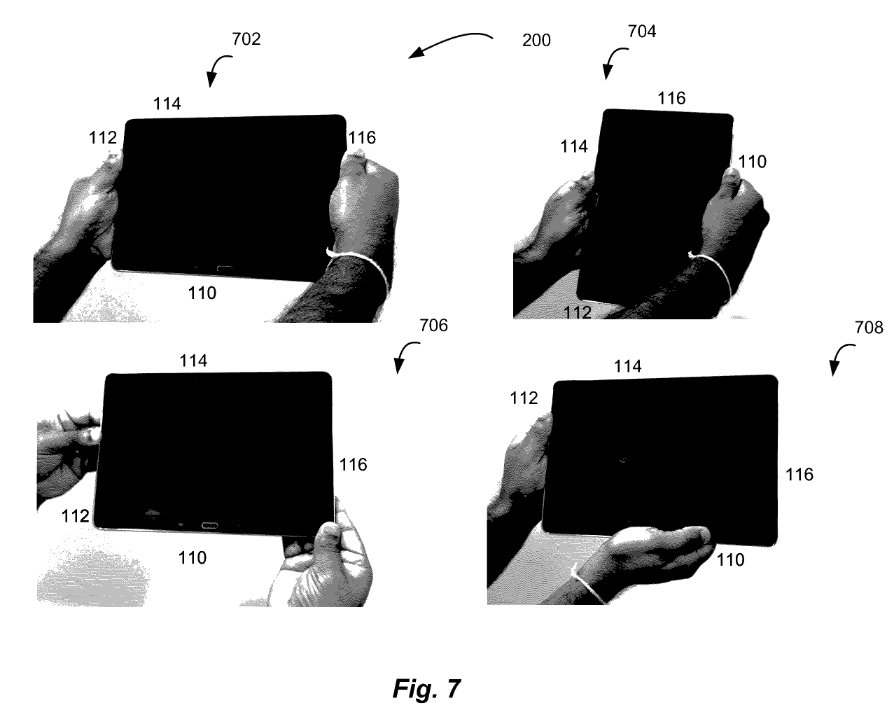

FIG. 7 illustrates different example views of a hand-held device configured with opportunistic measurements of a user's physiological context, in accordance with some embodiments. Views 702, 706, and 708 illustrate examples in which a user holds the device 200 in a landscape mode. View 704 illustrates an example in which a user holds the device 200 in a portrait mode. The example configuration of the electrodes on the hand-held device 200 may correspond to that referenced in FIG. 2.

For example, the user may hold the device 200 by its sides, thus providing desired contact with respective electrodes, such as electrodes 112 and 116 (as shown in FIG. 2 and view 702) or 110 and 114 (as shown in FIG. 2 and view 704) disposed around the perimeter of the back surface of the hand-held device.

However, as may be evident from views 706 and 708, the user may hold the device 200 touching two electrodes with one hand at the same time. For example, again referencing FIG. 2, the user may be touching electrode 112 with her left hand and touching electrodes 110 and 116 with her right hand, if she holds the device 200 as shown in view 706. In another example, the user may be touching a vertically disposed electrode 112 with her one hand and horizontally disposed electrode 110 with her other hand, as shown in view 708.

Following the example illustrated by view 706, an appropriate electrode pair may be selected for ECG sensing based on the embodiments described in reference to FIG. 6, such as determining the lowest impedance (or ETI voltage) between electrode pairs {112, 110} or {112, 116}. Further, based on orientation of the device 200, it may be determined that electrode 112 is on the left side of the user. Because the fingers of the same hand touch the electrodes {110, 116}, very low (e.g., below a threshold) body impedance (ETI voltage) between pair {110, 116} may be expected, and may fall outside the acceptable ETI range. Accordingly, pair {110, 116} may be discarded from the selection process.

With reference to view 708 (user touches electrodes 112 and 110), orientation of the device 200 may not be sufficient to identify a correct electrode pair. However, contact between electrode pair {112, 110} may be identified, using ETI voltage. Further, the electrode corresponding to a left side of the user's body may be identified for this pair (e.g., 112) using the orientation technique described in reference to FIG. 6. The foregoing described views are meant to be illustrative, and are not meant to be exhaustive of all possible manners a user may hold a hand-held device with one or both hands.

The embodiments described in reference to FIGS. 1-7 may provide the following advantages. The described embodiments may enable opportunistic, not just intentional, measurements of the user's physiological context (e.g., ECG sensing) in a hand-held device form factor. The described embodiments may enable identification of the electrode pair with desired skin contact for physiological context measurements, from an array of electrodes disposed on the surface (e.g., back side, bezel, and/or other portions of a casing) of a hand-held device. This significantly improves quality and reliability of the user's physiological context measurements. Further, individual electrodes may be connected with the correctly identified polarity to the input of an ECG amplifier, to derive a medically appropriate (Lead-1) ECG waveform, in any arbitrary orientation of the hand-held device. The described embodiments may enable injection and sampling of current from any combination of electrode pairs, thus providing for sensing ETI as well as ECG from any combination of electrode pairs. Adaptive noise cancellation of noise artifacts from an ECG signal induced in an opportunistic sensing of the user's physiological context may be employed, due to the use of synchronous ETI and ECG readings in any orientation of the hand-held device. Further, a number, shape, size, placement and layout of the electrodes disposed on the hand-held device may offer various opportunities for ECG sensing during casual everyday use of hand-held devices.

The described embodiments may enable several applications, such as in cardiac health monitoring, arrhythmia detection, normal or abnormal ECG classification, cardiac health trends, biometric authentication, and the like. ECG measurements may also be used for other applications, such as heart rate monitoring, emotional monitoring, stress detection, and the like.

The following paragraphs describe examples of various embodiments.

Example 1 is a hand-held device, comprising: a surface that includes two or more electrodes disposed on the surface to contact with portions of one or more hands of a user when the portions of the one or more hands are in contact with the surface while the user holds the hand-held device, to obtain signals to be used to determine a physiological context of the user; and circuitry coupled with the two or more electrodes to identify at least two of the two or more electrodes that are in contact with the user's respective portions of the one or more hands and, on identification, collect the signals to determine the physiological context using the identified electrodes while the contact between the portions of the one or more hands and the identified electrodes is maintained.

Example 2 may include the subject matter of Example 1, wherein the two or more electrodes are disposed substantially around a perimeter of the surface, to enable contact of the user's portions of the one or more hands with the at least two of the two or more electrodes while the user holds the hand-held device.

Example 3 may include the subject matter of Example 2, wherein the two or more electrodes comprise four electrodes having trapezoid-like shapes, to extend toward edges of the surface of the hand-held device to provide the contact of the user's portions of the one or more hands with the identified electrodes.

Example 4 may include the subject matter of Example 1, wherein the circuitry includes: a selection circuit block, to cause a first current of a determined value to be injected in one of the two or more electrodes, and sequentially select each of remaining electrodes, to cause a second current to flow through the one or more hands and a body of the user; a sensing circuit block coupled with the selection circuit block, to detect whether the second current flows through the body of the user in response to the injection of the first current; and a controller, coupled with the selection and sensing circuit blocks, to: based on a result of the detection, cause the selection circuit block to repeat the injection and sequential selection until the second current is detected, and identify the electrodes that form a closed circuit with the one or more hands and the body; and receive the physiological context from the identified electrodes.

Example 5 may include the subject matter of Example 4, wherein the circuitry is to perform identification of the electrodes on a continuous or periodic basis, to dynamically track holding of the hand-held device by the user's portions of the one or more hands.

Example 6 may include the subject matter of Example 4, wherein the circuitry further includes: a device orientation circuit block coupled with the controller, to determine an orientation of the hand-held device; and a physiological context collection circuit block having positive and negative inputs coupled with the selection circuit block, wherein the controller is further to: based on the determined orientation, identify an order of a hold of the user's one or more hands of the hand-held device relative to the identified electrodes; and based on the identification, connect one of the identified electrodes to the positive input of the physiological context collection circuit block, and connect another of the identified electrodes to the negative input of the physiological context collection circuit block, to collect the signals, wherein the signals comprise electrocardiographic (ECG) signals, wherein the physiological context collection circuit block is to receive the ECG signals from the identified electrodes.

Example 7 may include the subject matter of Example 6, wherein the controller is further to process the ECG signals to eliminate or reduce noise introduced by the user's hands' contact with the identified electrodes, wherein to eliminate or reduce noise includes to process an electrode-tissue impedance (ETI) signal between the electrodes.

Example 8 may include the subject matter of Example 4, wherein to identify the electrodes further includes to determine whether an electrode-tissue impedance (ETI) signal between the identified electrodes is below a contact threshold.

Example 9 may include the subject matter of Example 4, wherein to identify the electrodes further includes to identify a pair of electrodes among the two or more electrodes that corresponds to a lowest electrode-tissue impedance (ETI) signal between the electrodes.

Example 10 may include the subject matter of Example 1, wherein the circuitry further comprises an optical sensor coupled with at least one electrode to provide a photoplethysmogram (PPG) of the user.

Example 11 may include the subject matter of Example 1, wherein the circuitry is to provide the collected signals to an external device communicatively coupled with the hand-held device to determine the physiological context.

Example 12 may include the subject matter of Example 1, wherein the physiological context includes at least some of: electrocardiographic (ECG) data, photoplethysmographic (PPG) data, electromyographic (EMG) data, or electroencephalographic (EEG) data.

Example 13 may include any part of the subject matter of Examples 1 to 12, wherein the hand-held device is a tablet computer or a smartphone, wherein the surface comprises at least a part of: a bezel or a back side of the hand-held device, wherein the portions of one or more hands are selected from one of: fingers, palms, or wrists, and wherein the portions of one or more hands are disposed at least in part on the surface of the hand-held device to hold and interact with the hand-held device.

Example 14 is a hand-held device, comprising: a casing, having at least one surface that includes two or more electrodes disposed on the at least one surface to contact with portions of one or more hands of a user when the portions of the one or more hands are in contact with the surface while the user holds the hand-held device, to obtain signals to be used to determine a physiological context of the user; and circuitry coupled with the two or more electrodes to identify at least two of the two or more electrodes that are in contact with the user's respective portions of the one or more hands and, on identification, collect the signals to determine the physiological context using the identified electrodes while the contact between the portions of the one or more hands and the identified electrodes is maintained.

Example 15 may include the subject matter of Example 14, wherein the surface of the casing comprises at least a part of a back surface of the hand-held device and a bezel of the hand-held device, wherein the two or more electrodes comprise four electrodes having trapezoid-like shapes and disposed substantially around a perimeter of the back surface, to extend toward edges of the back surface of the hand-held device to provide the contact of the user's portions of the one or more hands with the at least identified electrodes.

Example 16 may include the subject matter of Example 15, wherein the circuitry is to: inject a first current of a determined value in one of the four electrodes; sequentially select each of remaining electrodes, to cause a second current to flow through the one or more hands and a body of the user; detect whether the second current flows through the body of the user in response to the injection of the first current; and, based on a result of the detection, identify the electrodes that form a closed circuit with the one or more hands and the body, wherein the identified electrodes provide the signals that are sensed from the respective portions of one or more hands of the user.

Example 17 may include the subject matter of Example 16, wherein the circuitry is further to: determine an orientation of the hand-held device; based on the determined orientation, identify an order of a hold of the user's one or more hands of the hand-held device relative to the identified electrodes; and based on the identification, connect the identified electrodes to respective inputs of the circuitry, to collect the physiological context, wherein the physiological context comprises electrocardiographic (ECG) data.

Example 18 may include any part of the subject matter of Examples 14 to 17, wherein the hand-held device comprises one of: a tablet computer or a smartphone.

Example 19 is a method for measurements of physiological context with a hand-held device, comprising: identifying, by a circuitry of a hand-held device, at least two of two or more electrodes disposed on the hand-held device and communicatively coupled with the circuitry, that are in contact with respective portions of one or more hands of a user while the user holds the hand-held device, to obtain signals to be used to determine a physiological context of the user; and, based on a result of the identification, collecting, by the circuitry, the signals to determine the physiological context using the identified electrodes while the contact between the portions of the one or more hands and the identified electrodes is maintained.

Example 20 may include the subject matter of Example 19, wherein identifying includes: injecting, by the circuitry, a first current of a determined value in one of the two or more electrodes; sequentially selecting, by the circuitry, each of remaining electrodes, to cause a second current to flow through the one or more hands and a body of the user; detecting, by the circuitry, whether the second current flows through the one or more hands and the body of the user in response to the injection of the first current; and based on a result of the detection, identifying, by the circuitry, the electrodes that form a closed circuit with the body.

Example 21 may include the subject matter of Example 19, further comprising: determining, by the circuitry, an orientation of the hand-held device; based on the determined orientation, identifying, by the circuitry, an order of a hold of the user's one or more hands of the hand-held device relative to the identified electrodes; and based on the identification, connecting, by the circuitry, the identified electrodes to respective inputs of the circuitry, to collect signals to determine the physiological context, wherein the signals comprise electrocardiographic (ECG) data signals.

Example 22 is a hand-held device, comprising: means for identifying at least two of two or more electrodes disposed on the hand-held device, that are in contact with respective portions of one or more hands of a user while the user holds the hand-held device, to obtain signals to be used to determine a physiological context of the user; and, means for collecting, based on a result of the identification, the signals to determine the physiological context using the identified electrodes while the contact between the portions of the one or more hands and the identified electrodes is maintained.

Example 23 may contain the subject matter of Example 22, wherein the means for identifying further include: means for injecting a first current of a determined value in one of the two or more electrodes; means for sequentially selecting each of remaining electrodes, to cause a second current to flow through the one or more hands and a body of the user; means for detecting whether the second current flows through the one or more hands and the body of the user in response to the injection of the first current; and means for identifying the electrodes that form a closed circuit with the body, based on a result of the detection.

Example 24 may contain the subject matter of Example 22, further comprising: means for determining an orientation of the hand-held device; means for identifying an order of a hold of the user's one or more hands of the hand-held device relative to the identified electrodes, based on the determined orientation; and means for connecting the identified electrodes to respective inputs of circuitry disposed in the hand-held device, to collect signals to determine the physiological context, wherein the signals comprise electrocardiographic (ECG) data signals, based on the identification.

Various operations are described as multiple discrete operations in turn, in a manner that is most helpful in understanding the claimed subject matter. However, the order of description should not be construed as to imply that these operations are necessarily order dependent. Embodiments of the present disclosure may be implemented into a system using any suitable hardware and/or software to configure as desired.

Although certain embodiments have been illustrated and described herein for purposes of description, a wide variety of alternate and/or equivalent embodiments or implementations calculated to achieve the same purposes may be substituted for the embodiments shown and described without departing from the scope of the present disclosure. This application is intended to cover any adaptations or variations of the embodiments discussed herein. Therefore, it is manifestly intended that embodiments described herein be limited only by the claims and the equivalents thereof.

What is claimed is:

1. A hand-held device, comprising:
a surface that includes two or more electrodes disposed on the surface to contact with portions of one or more hands of a user when the portions of the one or more hands are in contact with the surface while the user holds the hand-held device, to obtain signals to be used to determine a physiological context of the user; and
circuitry coupled with the two or more electrodes to identify at least two of the two or more electrodes that are in contact with the user's respective portions of the one or more hands and, on identification, collect the signals to determine the physiological context using the identified electrodes while the contact between the portions of the one or more hands and the identified electrodes is maintained.

2. The hand-held device of claim 1, wherein the two or more electrodes are disposed substantially around a perimeter of the surface, to enable contact of the user's portions of the one or more hands with the at least two of the two or more electrodes while the user holds the hand-held device.

3. The hand-held device of claim 2, wherein the two or more electrodes comprise four electrodes having trapezoid-like shapes, to extend toward edges of the surface of the hand-held device to provide the contact of the user's portions of the one or more hands with the identified electrodes.

4. The hand-held device of claim 1, wherein the circuitry includes:
a selection circuit block, to cause a first current of a determined value to be injected in one of the two or more electrodes, and sequentially select each of remaining electrodes, to cause a second current to flow through the one or more hands and a body of the user;
a sensing circuit block coupled with the selection circuit block, to detect whether the second current flows through the body of the user in response to the injection of the first current; and
a controller, coupled with the selection and sensing circuit blocks, to:
based on a result of the detection, cause the selection circuit block to repeat the injection and sequential selection until the second current is detected, and identify the electrodes that form a closed circuit with the one or more hands and the body; and
receive the physiological context from the identified electrodes.

5. The hand-held device of claim 4, wherein the circuitry is to perform identification of the electrodes on a continuous or periodic basis, to dynamically track holding of the hand-held device by the user's portions of the one or more hands.

6. The hand-held device of claim 4, wherein the circuitry further includes:
a device orientation circuit block coupled with the controller, to determine an orientation of the hand-held device; and
a physiological context collection circuit block having positive and negative inputs coupled with the selection circuit block,
wherein the controller is further to:
based on the determined orientation, identify an order of a hold of the user's one or more hands of the hand-held device relative to the identified electrodes; and
based on the identification, connect one of the identified electrodes to the positive input of the physiological context collection circuit block, and connect another of the identified electrodes to the negative input of the physiological context collection circuit block, to collect the signals, wherein the signals comprise electrocardiographic (ECG) signals, wherein the physiological context collection circuit block is to receive the ECG signals from the identified electrodes.

7. The hand-held device of claim 6, wherein the controller is further to process the ECG signals to eliminate or reduce noise introduced by the user's hands' contact with the identified electrodes, wherein to eliminate or reduce noise includes to process an electrode-tissue impedance (ETI) signal between the electrodes.

8. The hand-held device of claim 4, wherein to identify the electrodes further includes to determine whether an electrode-tissue impedance (ETI) signal between the identified electrodes is below a contact threshold.

9. The hand-held device of claim 4, wherein to identify the electrodes further includes to identify a pair of electrodes among the two or more electrodes that corresponds to a lowest electrode-tissue impedance (ETI) signal between the electrodes.

10. The hand-held device of claim 1, wherein the circuitry further comprises an optical sensor coupled with at least one electrode to provide a photoplethysmogram (PPG) of the user.

11. The hand-held device of claim 1, wherein the circuitry is to provide the collected signals to an external device communicatively coupled with the hand-held device to determine the physiological context.

12. The hand-held device of claim 1, wherein the physiological context includes at least some of: electrocardiographic (ECG) data, photoplethysmographic (PPG) data, electromyographic (EMG) data, or electroencephalographic (EEG) data.

13. The hand-held device of claim 1, wherein the hand-held device is a tablet computer or a smartphone, wherein the surface comprises at least a part of: a bezel or a back side of the hand-held device, wherein the portions of one or more hands are selected from one of: fingers, palms, or wrists, and wherein the portions of one or more hands are disposed at least in part on the surface of the hand-held device to hold and interact with the hand-held device.

14. A hand-held device, comprising:
a casing, having at least one surface that includes two or more electrodes disposed on the at least one surface to contact with portions of one or more hands of a user when the portions of the one or more hands are in contact with the surface while the user holds the hand-held device, to obtain signals to be used to determine a physiological context of the user; and
circuitry coupled with the two or more electrodes to identify at least two of the two or more electrodes that are in contact with the user's respective portions of the one or more hands and, on identification, collect the signals to determine the physiological context using the identified electrodes while the contact between the portions of the one or more hands and the identified electrodes is maintained.

15. The hand-held device of claim 14, wherein the surface of the casing comprises at least a part of a back surface of the hand-held device and a bezel of the hand-held device, wherein the two or more electrodes comprise four electrodes having trapezoid-like shapes and disposed substantially around a perimeter of the back surface, to extend toward edges of the back surface of the hand-held device to provide the contact of the user's portions of the one or more hands with the at least identified electrodes.

16. The hand-held device of claim 15, wherein the circuitry is to:
inject a first current of a determined value in one of the four electrodes;
sequentially select each of remaining electrodes, to cause a second current to flow through the one or more hands and a body of the user;
detect whether the second current flows through the body of the user in response to the injection of the first current; and
based on a result of the detection, identify the electrodes that form a closed circuit with the one or more hands and the body,
wherein the identified electrodes provide the signals that are sensed from the respective portions of one or more hands of the user.

17. The hand-held device of claim 16, wherein the circuitry is further to:
determine an orientation of the hand-held device;
based on the determined orientation, identify an order of a hold of the user's one or more hands of the hand-held device relative to the identified electrodes; and
based on the identification, connect the identified electrodes to respective inputs of the circuitry, to collect the physiological context, wherein the physiological context comprises electrocardiographic (ECG) data.

18. The hand-held device of claim 14, wherein the hand-held device comprises one of: a tablet computer or a smartphone.

19. A method, comprising:
identifying, by a circuitry of a hand-held device, at least two of two or more electrodes disposed on the hand-held device and communicatively coupled with the circuitry, that are in contact with respective portions of one or more hands of a user while the user holds the hand-held device, to obtain signals to be used to determine a physiological context of the user; and,
based on a result of the identification, collecting, by the circuitry, the signals to determine the physiological context using the identified electrodes while the contact between the portions of the one or more hands and the identified electrodes is maintained.

20. The method of claim 19, wherein identifying includes:
injecting, by the circuitry, a first current of a determined value in one of the two or more electrodes;
sequentially selecting, by the circuitry, each of remaining of the two or more electrodes, to cause a second current to flow through the one or more hands and a body of the user;
detecting, by the circuitry, whether the second current flows through the one or more hands and the body of the user in response to the injection of the first current; and
based on a result of the detection, identifying, by the circuitry, the electrodes that form a closed circuit with the body.

21. The method of claim 19, further comprising:
determining, by the circuitry, an orientation of the hand-held device;
based on the determined orientation, identifying, by the circuitry, an order of a hold of the user's one or more hands of the hand-held device relative to the identified electrodes; and
based on the identification, connecting, by the circuitry, the identified electrodes to respective inputs of the circuitry, to collect signals to determine the physiological context, wherein the signals comprise electrocardiographic (ECG) data signals.
